(19) United States
(12) Patent Application Publication
(10) Pub. No.: US 2019/0365527 A1
(43) Pub. Date: Dec. 5, 2019

(54) Reusable Delivery Apparatus for Delivering a Flexible Prosthesis to a Surgical Pocket

(71) Applicant: Bandula Wijay, Friendswood, TX (US)

(72) Inventor: Bandula Wijay, Friendswood, TX (US)

(21) Appl. No.: 15/997,381

(22) Filed: Jun. 4, 2018

(51) Int. Cl. A61F 2/12; A61B 17/34

(52) U.S. Cl. CPC A61F 2/12; A61B 2017/00796; A61F 2230/0071; A61B 17/3468

(57) ABSTRACT

The delivery device for a breast implant has the shape of a rectangular cross section also enables to surgeon to orient the implant appropriately so that the flat bottom is parallel to the direction of the surgical incision. As such when the implant is delivered it would orient itself in the same plane as the surgical incision and hence in a plane more parallel to the chest wall.

### FIELD OF THE INVENTION

This invention is related to a reusable apparatus and method for the delivery of a soft flexible prosthetic implant such as a silicone breast implant into the surgical pocket and also having the option for packaging the implant within the delivery apparatus, in sterile condition, for use without having to transfer the implant into a separate apparatus for implantation, thereby providing a contamination free path for implantation or for providing a reusable, re-serializable delivery device apparatus to deliver a soft flexible prosthetic implant.

### BACKGROUND OF THE INVENTION

Silicone implants and other soft and flexible implants are placed within body tissue for both physical augmentations for aesthetic appearance and or for therapeutic purposes. In most instances the surgeon creates an incision on the body surface through which the implant is introduced either manually or using a delivery apparatus. When it is introduced manually, considerable handling of the implant takes place which exposes the implant to mechanical damage and or contamination. Implant failure post implantation due to mechanical damage and infections due to excessive handling contamination are well documented in the literature. During the last decade several devices have been developed to prevent or minimize these procedure failures due to damage or infections.

One of the most common devices known as Keller Funnel as described in U.S. Pat. No. 8,211,173 B2 is an example of such a delivery apparatus. The Keller funnel is essentially a flat flexible plastic cone, similar to a cake decorating cone, with a distal open end for the delivery of the implant and a large proximal opening to introduce the implant into the lay flat cone. Once the implant is placed in the flexible plastic cone and some lubricant is added, the surgeon squeezes the proximal end of the plastic cone in order to extrude the implant out from the cone's distal end. Most surgeons will have to use considerable effort and both hands to squeeze the implant out of the Keller funnel and requiring considerable effort. In order to minimize this effort, surgeons will cut open the distal end to increase the size of the distal opening and thereby reducing the effort needed to extrude out the implant.

One of the biggest drawbacks encountered during delivery of an implant using the Keller funnel is that the implant needs to be oriented appropriately in the delivery apparatus. While the funnel is substantially flat and the implant is bun like in nature, once the circularly shaped implant is placed within it, the implant takes a symmetrical spherical shape and hence loses its bun like shape and the intended orientation that is very important for the appropriate placement of the implant in the pocket. To prevent this from happening the surgeon has to be extra conscious in orienting the cone and in lining up the implant with the surgical pocket during the delivery process, which rather complicated and often ineffective.

Another disadvantage of the Keller Funnel is that when the implant is placed inside the delivery envelope, it takes a conical shape and therefore when the implant is delivered the proximal end of the implant tends to fold inwards forming a crease which can cause structural damage to the silicone envelop of the implant. This type of trauma to the implant wall can lead to implant failure, infection and or capsular contracture.

In a typical breast augmentation procedure the surgeon creates a small incision in the periareolar, inframammary or transaxillary regions. The size of the incision depends on the size and volume of the implant been implanted. It is preferred that the incision is as small as possible to prevent a large scar on the skin surface. Through the incision, surgeon creates an appropriate pocket into which the implant will be placed. The incision is always linear and the pocket created is typically two dimensional in nature. Breast implants are typically circular in nature and are “bun” shaped having a thickness of 1-2 cm and the bottom side is flat in nature while the top side has a convex surface. The implant therefore must be appropriately oriented so that the implant sits parallel to the floor of the pocket created by the surgeon. If the implant is not properly oriented the appearance of the breast augmentation will not have the natural appearance. The design in the Keller Funnel fails to provide the surgeon the ability to properly orient the implant such that it sits parallel to the floor of the pocket created by the surgeon.

Another disadvantage of the flexible cone type delivery methods is that the distal opening of the funnel can tear under pressure. Often reinforcements for the distal opening are proposed as in U.S. Pat. No. 8,211,173.

### SUMMARY OF THE INVENTION

The present invention is a delivery apparatus and method for a reusable implant delivery apparatus which is constructed from metal or durable polymer that can be cleaned and reserialized.

Another objective of the present invention is to provide a means to package the implant in the delivery apparatus in sterile condition so that it can be delivered to the surgical pocket without having to transfer the implant to a separate delivery apparatus, thereby eliminating handling and contamination.

Therefore present invention provides a design such that the implant can be sterilized in the delivery apparatus which has appropriate openings for sterilization gases to flow in and also has openings in the apparatus to introduce fluids prior to its use, either for lubrication and washing the implant as needed or to provide hydraulic pressure within it during extrusion of the implant.

A delivery device is provided such that the implant can be delivered with proper orientation and with minimum force. Thereby the fluid filled implants of different volumes can be extruded out through an appropriate delivery spout into the surgical pocket without using surgeon's fingers to push it through the skin opening.

A universal delivery device is provided that is capable of delivering implants of different sizes from different manufacturers into a surgical pocket when the implant is not packaged with the delivery device.

The proposed device and the packaging method enables appropriate orientation of the implant and at the same time requires a lesser delivery force to extrude the implant out of the delivery device. It also can be provided in a sterile form with the implant where no transfer is needed from the package to an additional delivery device and hence minimizing any contamination or without an implant where the surgeon can select an implant of his/her choice. Also provided are the openings for adding lubricants and or washing solutions that may be desired by the attending surgeon.

As the delivery apparatus has a generally a rectangular shape and a conical distal portion that guides the implant into the surgical pocket will prevent the implant from folding and causing structural damage to the silicone envelop of the implant encountered by the current state of the art delivery apparatus.

The delivery device in the present invention is generally rigid in nature and is designed to maintain its rectangular shape without significant deformation to its character. The materials used for the construction can be stainless steel, molded Nylon, molded polycarbonate, molded Polyethylene Terephthalate (PET) or similar polymer material and can be made by molding, vacuum forming or 3D printing using rigid or semi-rigid clear or opaque plastics.

Many variations of the present invention are possible and the description herein is meant to disclose and discuss the general principles associated with the invention and other variations can be developed by one knowledgeable in the state of the art. Various details are left out for clarity which is part and parcel of the present invention.

The breast implant as discussed earlier is generally bun shaped having a circular nature and a smaller height as compared to its diameter as shown in FIG. 1. The following Table 1 is a typical example of an implant with a moderate projection. Other models with higher projections and larger diameters are commercially available.

TABLE 1

| Vol. | Diam. | Projection |
|---|---|---|
| 125 cc | 9.5 cm | 3.0 cm |
| 150 cc | 10.0 cm | 3.1 cm |
| 175 cc | 10.6 cm | 3.3 cm |
| 200 cc | 11.0 cm | 3.4 cm |
| 225 cc | 11.5 cm | 3.5 cm |
| 250 cc | 11.9 cm | 3.6 cm |
| 275 cc | 12.3 cm | 3.7 cm |
| 300 cc | 12.6 cm | 3.7 cm |
| 325 cc | 13.0 cm | 3.8 cm |
| 350 cc | 13.3 cm | 3.9 cm |
| 375 cc | 13.6 cm | 4.0 cm |
| 425 cc | 14.2 cm | 4.1 cm |
| 475 cc | 14.8 cm | 4.2 cm |

In any given situation the volume of the implant is constant. For example the implant from the above table having a volume of 475 cc has a diameter of 14.8 cm and a projection (height) of 4.2 cm. When this is placed in a conical delivery device as shown in FIG. 2, the implant will fill the cone as shown in FIG. 2, where the diameter of the implant at the top (d) and the height (h) will be dependent on its initial volume of 475 cc. This relationship can be expressed mathematically as;

475 cc=V=⅓πr²h= 1/12πd²h (1)

As shown in FIG. 3, the pressure (P) exerted by the manual squeezing of the flexible cone causes the implant to be extruded out of the cone. This pressure is created by the force generated by the surgeon's palms and is equal to the force (F) and the area (A) of the face of the implant in the delivery cone. If the equation is rearranged the Pressure P generated to extrude the implant can be expressed as;

P=F·h/3V (2)

Or P is proportional to h/3

Now referring to FIG. 4, where the delivery device has more of a rectangular nature rather than a conical nature, the calculations can be repeated. Let's consider the dimensions in FIG. 4 where the rectangular cross section has a length (L) and a width (W). Let's note that the height correspondingly to be (h). Now calculating the volume of the implant, as in the previous example of 475 cc;

475cc=V=L·W·h=A·h (3)

where A=L·W
By rearranging the equation (3) above;

Area=A=V/h

Therefore Pressure generated P=F/A=F·h/V (4)

Or P is proportional to h

From this analysis it is quite evident that the pressure generated from a constant force (F) to extrude the implant out from a conical shape for a given volume is three times smaller than for a rectangular delivery device designed to deliver an implant of the same volume.

The delivery device has the shape of a rectangular cross section also enables to surgeon to orient the implant appropriately so that the flat bottom is parallel to the direction of the surgical incision. As such when the implant is delivered it would orient itself in the same plane as the surgical incision and hence in a plane more parallel to the chest wall. Therefor no attention is needed by the surgeon to be especially careful of the orientation of the implant after delivery which saves time and additional manipulations post implantation.

There are ports to introduce lubricants and fluids for rinsing while providing means for sterilization gases to enter and exit freely during the sterilization process. The vent feature provided will allow the surgeon to vent any air out of the system and thereby enhance the pressurization needed to extrude the implant, as any air present would simply compress without providing enhanced traction for the implant.

### BRIEF DESCRIPTION OF THE DRAWINGS

FIG. 1. Shows the typical shape of a breast implant

FIG. 2. Shows the current delivery device (Keller Funnel) that is used in the delivery of breast implants

FIG. 3. Shows the current delivery device with the proximal end closed and with the implant in its delivery position having a height (h) and a diameter at its face of (d)

FIG. 4a. Shows a side view diagrammatic configuration of the proposed implant delivery device having a rectangular base with a height of implant (h), a push plunger and a layer of fluid between the implant and the plunger

FIG. 4b. Shows a plan view diagrammatic configuration of the proposed implant delivery device having a rectangular base with a length (L), width (W)

FIG. 5. Shows the proposed embodiment having a general rectangular cavity having a spout at its distal end and a plunger placed proximally to push and extrude the implant out of the delivery device.

FIG. 6. Shows a cross section of the embodiment at Section A-A, showing that the cross section has a flat base and a convex top surface while having generally parallel side walls.

FIG. 7. Shows the method wherein the implant delivery device containing the implant is packaged in a blister package having a peel off sheath sealed on to it. By removing the peel off sheath, the delivery device can be removed from the package.

FIG. 8 shows how the surgeons hand and fingers are used to push the plunger into the apparatus to extrude the implant out.

### DETAILED DESCRIPTION OF THE PREFERRED EMBODIMENTS

The description herein describes an embodiment representing and functioning to achieve the objectives set forth in the invention and is not the only form a device can be constructed and utilized. The description sets forth the function and the design features of the various components and the method of operation of the proposed Sterile Packaging Method and an Implant Delivery Apparatus and variations can be achieved by anyone with similar skills with various other embodiments of similar function.

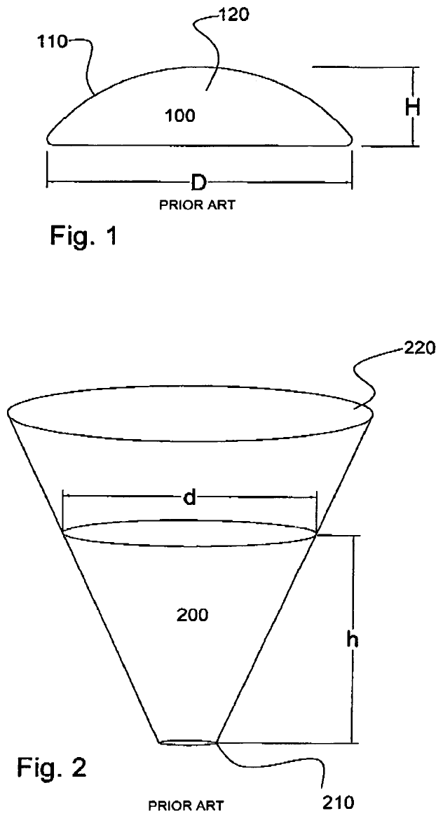

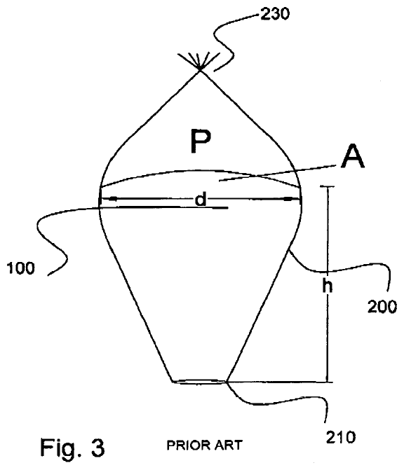

FIG. 1 shows a typical breast implant (100) having a base diameter of “D” and a height of “H”. The implant has a general “bun” like shape and is soft and fluid in nature. The outer skin (110) of the implant is typically made from a silicone membrane and the implant contains a more fluidic silicone gel material (120) inside the outer capsule. Breast implants are available in a variety of shapes and sizes but all of them generally have the shape and format described in FIG. 1.

FIG. 2 shows the current apparatus for the delivery of breast implants into surgical pockets. The surgeon places the breast implant into the cone (200), carefully orienting it appropriately and manually squeezes into to the surgical pocket similar to squeezing cake icing on to a cake.

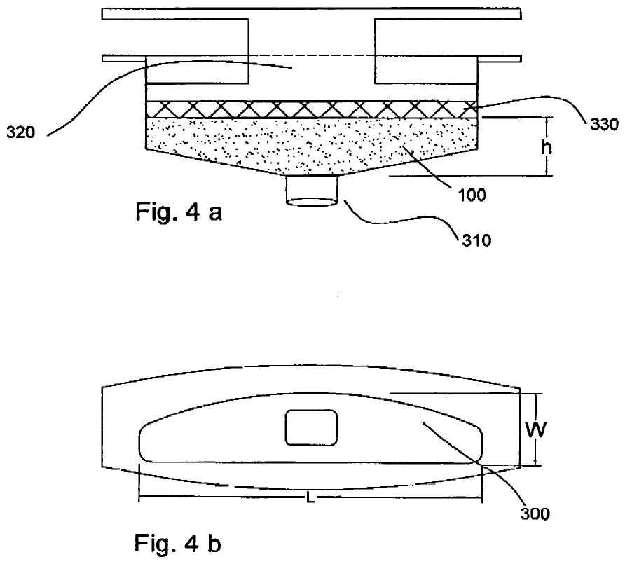

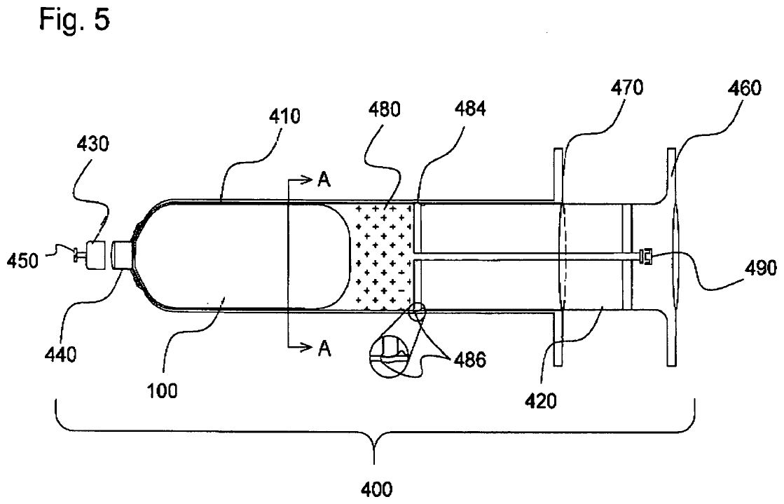

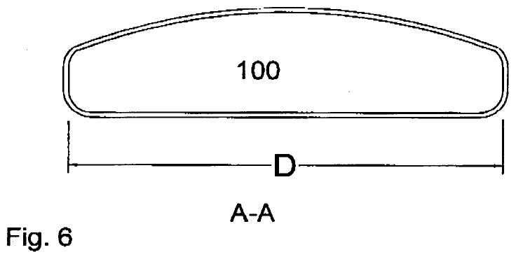

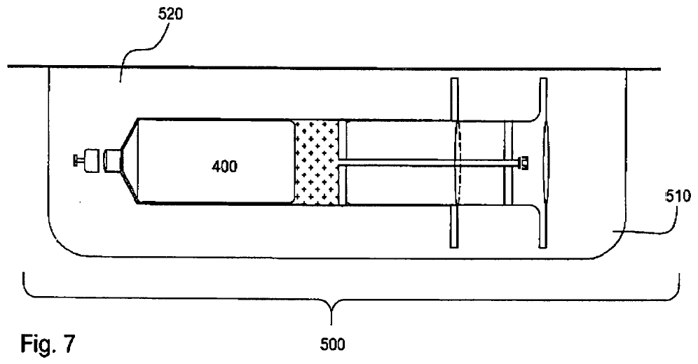

The diagram in FIG. 4a and FIG. 4b shows the proposed implant delivery device having a rectangular cavity (300) where the silicone breast implant will be placed and a spout (310) which has generally rectangular tubular shape with smooth corners and a driver piston plunger (320) to push the implant out through the exit spout (310). The space unoccupied by the implant (100) in the cavity (300) is filled with fluid (330). The fluid can be any fluid similar to saline/lubricant mixture or saline containing antibiotics or can also be a solution of betadine. A detailed design of the proposed implant delivery device is shown in FIG. 5. The delivery apparatus (400) consists of three main components; the implant holder (410), the plunger (420) the cap (430). The implant (100) is placed inside the implant holder (410) until it is in contact with throat of the spout (440). The spout 440) can be made in several different sizes suitable to fit the specific surgical slit. The spout is attached to the implant holder with a suitable means such as a twist lock, or snap lock means that are common to attaching a spout into a body. The interface between the spout and the implant holder will be smooth to prevent any hang up of the implant when it is pushed through the spout. The cap (430) has a port that contains a luer attachment (450) which can be connected to a syringe in order to add lubricants or saline into the implant holder (410). The port (450) also will enable the surgeon to bleed any air that is left in the apparatus before the implant is extruded out. The cap (430) fits tightly on to the spout (440) but can be removed with ease when not in use. The open port (450) allows the sterilization gases to enter the apparatus during sterilization. The plunger (420) slides within the implant holder (410). The plunger has its distal end dimension slightly larger than the inside dimension of the implant holder as shown in (484) so that it provides a tight hermetic seal between the plunger and the implant holder. The face of the plunger has a concave nature (486) so in case when the implant is in contact with the face of the plunger, the curvature will guide the implant radially inwards than allowing it into the gap between the plungers' face and the implant holder. This will prevent any damage that can occur if the implant would get caught up in the gap between the plunger and implant holder. Proximally the plunger has an expanded collar (460) having a concave nature that would fit to the palm of the surgeon's hand. Similarly the implant holder (410) also has a collar (470) at its proximal end. The user will hold the implant delivery device with one hand and push the plunger with the other hand, and at the same time will use the two collars as means to grab and pull the collars towards each other in order to push the plunger (420) into the implant holder (410) as shown in FIG. 7. The cross section view (A-A) of the implant holder is shown in FIG. 6 where the bottom side of the implant holder is flat and the top side of the implant holder has a convex nature so that the breast implant would fit nicely into the cavity while orienting itself for proper delivery.

When the implant delivery apparatus is provided without an implant (FIG. 5), the surgeon will remove the plunger (420) and place the implant in the implant holder (410). Due to its rectangular shape the implant will fit easily in and orients itself in a manner that facilitates the proper placement of the implant in the surgical pocket. The plunger (420) will be placed back into the holder (410) after few cc of saline or betadine is added to the implant holder. Any added fluid that will flow to the distal end of the delivery device can be bled out through the luer port (450). Similarly the luer port (450) can be used to introduce saline or betadine solution or any lubricants the surgeon may feel would assist the extrusion of the implant out of the delivery device (400).

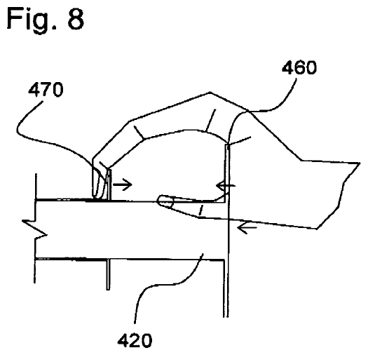

The implant (100) can be supplied sterile within the delivery device (400) as shown in FIG. 8 in a sterile pack (500) which is made of two parts, the tray (510) and the lid (520), which is sealed in place at the factory. The lid (520) is peeled off to retrieve the delivery device (400). This will make it convenient for the surgeon to introduce the implant into the surgical cavity without having the need to transfer the implant from the sterile package into the delivery device. This method will also eliminate any potential contamination that might occur during the any handling process. The saline for washing or rinsing the implant and any lubricant needed can be added through the luer port (490) and luer port (450). The delivery device therefore will be disposable and as such each individual breast implant will be packaged in a delivery apparatus that is built to fit the exact implant shape and size making the delivery process more efficient, sterile and minimum handling.

I claim:

1. An implant delivery assembly, comprising:
a housing comprising facing opposed sides that conform to opposed sides of an implant when the implant is in a relaxed condition to orient the implant as the implant is moved to a tapered end of said housing leading to an outlet for delivery into an incision in a patient;
an applicator associated with said housing to urge said implant through said outlet.

2. The assembly of claim 1, wherein:
one of said opposed sides is planar.

3. The assembly of claim 1, wherein:
one of said opposed sides is curved.

4. The assembly of claim 1, wherein:
said housing is substantially a quadrilateral in section view.

5. The assembly of claim 1, wherein:
said outlet size is variable with one of a plurality of replaceable spouts mounted to said outlet.

6. The assembly of claim 1, wherein:
said housing further comprises a fluid between the implant and said applicator.

7. The assembly of claim 1, wherein:
said housing with the implant therein and said applicator attached are packaged in an assembled sterile condition in a common package.

8. The assembly of claim 5, wherein:
said replaceable spouts further comprise a removable luer connection for injection of fluids before the implant is forced from said outlet.

9. The assembly of claim 1, wherein:
said applicator further comprises a hand operable plunger selectively pushed into said housing using a flange on said housing as a finger grip.

10. The assembly of claim 9, wherein:
said plunger comprises a detent in said housing to hold said plunger in a retracted position against a predetermined force.

11. The assembly of claim 1, wherein:
said housing orients the implant on a side of said implant.

12. The assembly of claim 9, wherein:
said plunger having a passage therethrough extending out of said housing for adding fluid to a space between said plunger and the implant.

13. The assembly of claim 1, wherein:
said opposed sides orient the implant for passage through said outlet and into the incision.

14. The assembly of claim 2, wherein:
another of said opposed sides is curved.

15. The assembly of claim 14, wherein:
said housing is substantially a quadrilateral in section view.

16. The assembly of claim 15, wherein:
said outlet size is variable with one of a plurality of replaceable spouts mounted to said outlet.

17. The assembly of claim 15, wherein:
said housing further comprises a fluid between the implant and said applicator.

18. The assembly of claim 17, wherein:
said housing with the implant therein and said applicator attached are packaged in an assembled sterile condition in a common package.

19. The assembly of claim 16, wherein:
said replaceable spouts further comprise a removable luer connection for injection of fluids before the implant is forced from said outlet.

20. The assembly of claim 15, wherein:
said applicator further comprises a hand operable plunger selectively pushed into said housing using a flange on said housing as a finger grip.

21. The assembly of claim 20, wherein:
said plunger comprises a detent in said housing to hold said plunger in a retracted position against a predetermined force.

22. The assembly of claim 21, wherein:
said housing orients the implant on a side of said implant.

23. The assembly of claim 21, wherein:
said plunger having a passage therethrough extending out of said housing for adding fluid to a space between said plunger and the implant.

24. The assembly of claim 22, wherein:
said opposed sides orient the implant for passage through said outlet and into the incision.

25. The assembly of claim 1, further comprising:
a breast implant with opposed sides conforming to the shape of said facing opposed sides of said housing.

26. The assembly of claim 1, wherein:
a predetermined size of said housing allows oriented delivery of breast implants of different sizes.
